(19) United States
(12) Patent Application Publication
(10) Pub. No.: US 2022/0040366 A1
(43) Pub. Date: Feb. 10, 2022

(54) Multi-Mode Lighting Device

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/509,877

(22) Filed: Oct. 25, 2021

Related U.S. Application Data
(63) Continuation-in-part of application No. 17/148,277, filed on Jan. 13, 2021, now Pat. No. 11,191,863.
Application No. 17/148,277 is a continuation-in-part of application No. 17/094,567, filed on Nov. 10, 2020, now Pat. No. 11,103,612.
Application No. 17/094,567 is a continuation-in-part of application No. 16/180,416, filed on Nov. 5, 2018, now Pat. No. 10,874,762.

(51) Int. Cl. A61L 9/20; B01D 46/00

(52) U.S. Cl. CPC A61L 9/205; A61L 2209/14; A61L 2209/12; B01D 46/0028

(57) ABSTRACT

A multi-mode lighting device includes a visible light source configured to emit a visible light and a controlling mechanism. The controlling mechanism is configured to operate the lighting device in at least two operational modes: a regular mode and a therapeutic mode. In the regular mode, the controlling mechanism operates the visible light source for general illumination. In the therapeutic mode, the controlling mechanism is configured to either flash the visible light source at a frequency in a frequency range of 35˜45 Hz or generate an audible sound at a frequency in a frequency range of 35˜45 Hz, or both. Some embodiments may support additionally a germicidal lighting mode, and some other embodiments with continuous photocatalyst-based air filtering and sanitization.

### CROSS REFERENCE TO RELATED PATENT APPLICATIONS

The present disclosure is a continuation-in-part (CIP) of U.S. patent application Ser. No. 17/148,277, filed 13 Jan. 2021, which is a CIP of U.S. patent application Ser. No. 17/094,567, filed 10 Nov. 2020, which is a CIP of U.S. patent application Ser. No. 16/180,416, filed 5 Nov. 2018 and issued as U.S. Pat. No. 10,874,762 on 29 Dec. 2020, the contents of which being incorporated by reference in their entirety.

### BACKGROUND

### Technical Field

The present disclosure pertains to the field of lighting devices and, more specifically, proposes a multi-mode lighting device.

### Description of Related Art

In U.S. patent application Ser. No. 17/148,277, an antiviral air-filtering lighting device includes an air-permeable air filter, a visible light source, a driver, and an air circulation mechanism. The air filter diffuses a visible light emitted from the visible light source and includes an air inlet port. The air filter is coated with a visible-light activatable antiviral photocatalytic coating. The visible light source is disposed inside the air filter to shine its light through the air filter to activate the visible-light activatable antiviral photocatalytic coating on the air filter. The air circulation mechanism sucks an ambient air from outside the lighting device and forces the air through the air filter. The air filter traps airborne microbials on the surface having the visible-light activatable antiviral photocatalytic coating. A light emitted by the first visible light source activates a photocatalyst material in the visible-light activatable antiviral photocatalytic coating, and the airborne microbials trapped by the air filter are killed or deactivated by the activated photocatalyst material.

Recent studies have found that visual and audible stimuli at 40 Hz to a subject has positive effects in treating Alzheimer's and related diseases. The present disclosure expands the functionality of the U.S. patent application Ser. No. 17/148,277 by incorporating the functionality of generating 40 Hz stimuli via a light or a sound or both, and supporting at least two operational modes, a regular lighting mode and a therapeutic lighting mode, during which the 40 Hz stimuli are generated. A more generalized multi-mode lighting device and a multi-mode lighting device adaptor are also proposed.

### SUMMARY

In one aspect, the light device comprises a first visible light source configured to emit a visible light, an air filter coated with an antiviral photocatalyst material, and an air circulation mechanism. The air circulation mechanism sucks an ambient air from outside the lighting device to force the air through the air filter, and the air filter traps airborne microbials as the air passing through. The visible light emitted by the first visible light source activates a photocatalyst material on the air filter. Lastly, the activated photocatalyst material kills or deactivates the airborne microbials trapped on the air filter.

In some embodiments, the lighting device further comprises an air inlet port, and the air circulation mechanism is disposed near the air inlet port.

In some embodiments, the first visible light source is disposed inside the air filter such that its light shines through the air filter to activate the antiviral photocatalyst material on the air filter. Moreover, the air filter diffuses the visible light emitted from the first visible light source. In other words, the air filter also functions as a diffuser to the first visible light source.

In some embodiments, the lighting device comprises no ultraviolet (UV) light source or infrared (IR) light source. In some other embodiments, the lighting device may include UV light source.

In some embodiments, the air filter requires no frame to house the first visible light source. The air filter can support its own structure without any additional frame internally or externally.

As stated earlier, the air filter does not contain or otherwise require any frame to house the first visible light source. However, under some situations, it may be beneficial to have an external housing to protect the air filter from damage. Thus, in some embodiments, the present disclosure further comprises a housing to house the air filter and the air circulation mechanism, and there are openings on the housing for the air to exit out of the lighting device.

In some embodiments, the antiviral photocatalytic coating on the air filter contains titanium oxide (TiO₂). In some other embodiments, the antiviral photocatalyst material coated on the air filter may contain titanium dioxide (TiO₂) and at least one metal photocatalyst material such as silver, gold, copper, zinc, nickel, or a combination thereof. These metals when embedded in the photocatalyst are known to enhance the photocatalytic activity with visible light. Some photocatalytic coating may contain more than one type of metals for a better photocatalytic effectiveness.

In some embodiments, the air circulation mechanism is a fan. It is foreseeable to have more than one fans to increase the airflow. It is also foreseeable to use a non-fan style air circulation mechanism.

In some embodiments, the first visible light source comprises one or more light emitting diodes (LEDs) each emitting the visible light.

In some embodiments, the first visible light source further comprises a second visible light source and a third visible light source, wherein a color temperature of the second visible light source is higher than a color temperature of the third visible light source. A light source with a higher color temperature tends to provide a stronger circadian stimulus and is thus suitable for daytime use. In comparison, a light source with a lower color temperature tends to provide a lesser circadian stimulus and is thus suitable for nighttime use. By having both color temperatures available, the present disclosure affords the user to choose a color temperature according to the circadian stimulus the user wants to receive.

In some embodiment, the lighting device further comprises a color-tuning controller. The color-tuning controller is configured to tune a color temperature of the first visible light source by mixing a combination ratio of the color temperatures of the second visible light source and the third visible light source, either manually or automatically according to a circadian schedule. With this functionality, rather than picking between two color temperatures only (from the second light source and the third light source), the user may now be able to select any color temperature between the color temperature of the second light source and the color temperature of the third light source.

In some embodiments, the lighting device further comprises a controlling mechanism for generating 40 Hz stimuli. The controlling mechanism is configured to either flash the light output of the first visible light source at a frequency in a frequency range of 35˜45 Hz or generates an audible sound at a frequency in a frequency range of 35˜45 Hz, or both. When the flashing light and/or the audible sound at 35˜45 Hz is perceived by a subject, it will induce synchronized gamma oscillations in at least one brain region of the subject. This may be used for treating people with dementia or Alzheimer's disease. The stimulus frequency at 40 Hz seems to have the best effect in inducing synchronized gamma oscillations in the brain.

In some embodiments, the controlling mechanism is configured to operate the lighting device in at least two operational modes: a regular mode and a therapeutic mode. In the regular mode, the controlling mechanism turning on the first visible light source as general illumination. In the therapeutic mode, the controlling mechanism is configured to either flash the light output of the first visible light source at a frequency in a frequency range of 35˜45 Hz or generate an audible sound at a frequency in a frequency range of 35˜45 Hz, or both. The regular mode and the therapeutic mode are not necessarily mutually exclusive. For example, if a controlling mechanism only generates a sound at 40 Hz frequency without flashing the light output of the first light source, then the controlling mechanism may operate the lighting device in both the regular mode (when the first light source is on) and the therapeutic mode (when a sound at 40 Hz frequency is generated) simultaneously.

The flashing of the visible light source may be realized through different means. In some embodiments, the controlling mechanism flashes the light output of the first visible light source by turning on/off the first visible light source at a frequency in a frequency range of 35˜45 Hz. In some other embodiments, the controlling mechanism flashes the light output of the first visible light source by alternating the light output level of the first visible light source between two different levels at a frequency in a frequency range of 35˜45 Hz, for example, between 50% and 100%. In some other embodiments, the controlling mechanism flashes the light output of the visible light source by alternating the color temperature of the first visible light source between two different color temperatures at a frequency in a frequency range of 35˜45 Hz. It is foreseeable to combine different means of flashing the light output of the first visible light source to create a stronger 40 Hz visual stimuli to a subject for inducing stronger synchronized gamma oscillations in subject's brain.

In some embodiments, the light device further includes a sound generator. The controlling mechanism generates an audible sound at a frequency in a frequency range of 35˜45 Hz via the sound generator. The sound generator may be a standalone device or a component of a controller circuit for implementing the controlling mechanism.

In another aspect of the present disclosure, the lighting device may include a visible light source configured to emit a visible light and a controlling mechanism. The controlling mechanism is configured to either flash the light output of the visible light source at a frequency in a frequency range of 35˜45 Hz or generate an audible sound at a frequency in a frequency range of 35˜45 Hz, or both.

In some embodiments, the controlling mechanism is configured to operate the lighting device in at least two operational modes: a regular mode and a therapeutic mode. In the regular mode, the controlling mechanism turning on the first visible light source as general illumination. In the therapeutic mode, the controlling mechanism is configured to either flash the light output of the first visible light source at a frequency in a frequency range of 35˜45 Hz or generate an audible sound at a frequency in a frequency range of 35˜45 Hz, or both. The regular mode and the therapeutic mode are not necessarily mutually exclusive. For example, if a controlling mechanism only generates a sound at 40 Hz frequency without flashing the light output of the first light source, then the controlling mechanism may operate the lighting device in both the regular mode (when the first light source is on) and the therapeutic mode (when a sound at 40 Hz frequency is generated) simultaneously.

In some embodiments, the controlling mechanism flashes the light output of the first visible light source by turning on/off the first visible light source at a frequency in a frequency range of 35˜45 Hz. In some other embodiments, the controlling mechanism flashes the light output of the first visible light source by alternating the light output level of the first light source between two different levels at a frequency in a frequency range of 35˜45 Hz. In some other embodiments, the controlling mechanism flashes the light output of the first visible light source by alternating the color temperature of the first visible light source between two different color temperatures at a frequency in a frequency range of 35˜45 Hz. It is foreseeable to combine different means of flashing the light output of the first visible light source to create a stronger 40 Hz visual stimuli to a subject.

In some embodiments, the light device further includes a sound generator. The controlling mechanism generates an audible sound at a frequency in a frequency range of 35˜45 Hz via the sound generator. The sound generator may be a standalone device or a component of a controller circuit for implementing the controlling mechanism.

In some embodiments, the lighting device further includes a second light source configured to emit a light in a wavelength range of 190 nm˜420 nm. The controlling mechanism is configured to operate the lighting device in at least three operational modes: a regular mode, a therapeutic mode, and a germicidal mode. In the regular mode, the controlling mechanism turning on the first visible light source as general illumination. In the therapeutic mode, the controlling mechanism is configured to either flash the light output of the first visible light source at a frequency in a frequency range of 35˜45 Hz or generate an audible sound at a frequency in a frequency range of 35˜45 Hz, or both. In the germicidal mode, the controlling mechanism turning on the second light source as germicidal irradiation. To prevent UV light exposure, there should be no occupants in the space during the germicidal mode, and consequently it may not be necessary to turn on the first visible light source for general illumination nor to generate any 40 Hz stimuli during the germicidal mode. It is foreseeable, however, there may be situation where the first visible light still needs to be on, say, for safety reason, during the germicidal mode. Therefore, it is not required for the first visible light mode to be turned off during the germicidal lighting mode. Conversely, if the second light source is configured to emit a UV irradiation dosage less than American Conference of Governmental Industrial Hygienists (ACGIH)-specified Threshold Limit Values (TLVs), then it is safe to keep the second light source on during the regular mode or the therapeutic mode. In this case, the lighting device may operate both the regular mode and the germicidal mode simultaneously, or the therapeutic mode and the germicidal mode simultaneously. In other words, the germicidal mode is not necessarily mutually exclusive to the regular mode or the therapeutic mode.

In some embodiment, the light output of the second light source is confined within the lighting device. In this case, there is no UV irradiation emitting out of the lighting device. Therefore, it is safe to operate the second light source all the time. Subsequently, in some embodiments, the controlling mechanism is further configured to always operate the lighting device. With these embodiments, they may support one of the following mixed operational modes:

1. germicidal mode

2. germicidal mode+regular mode

3. germicidal mode+therapeutic mode, and even

4. germicidal mode+regular mode+therapeutic mode

In this case, the controlling mechanism always operates the lighting device in the germicidal mode continuously. At times, the controlling mechanism may also operate the lighting device in the regular mode or the therapeutic mode or both. For example, if the controlling mechanism would only generate an audible sound at 40 Hz frequency (i.e., without flashing the light output of the first light source), then the controlling mechanism may operate the lighting device in the regular mode (with first light source on), the therapeutic mode (with the audible sound at 40 Hz frequency on), and the germicidal mode (with the second light source on) simultaneously.

In another aspect of the present disclosure, the lighting device adaptor for an external lighting device may include an electrical input port, an electrical output port, and a controller circuit. The electrical input port is connected an external power source, and the electrical output port is connected to an electrical input port of an external lighting device for providing power to the external lighting device. Moreover, the controller circuit is configured to either flash the light output of the external lighting device at a frequency in a frequency range of 35˜45 Hz or generate an audible sound at a frequency in a frequency range of 35˜45 Hz, or both. This lighting device adaptor doesn't have its own light source. It is meant to be used with an external lighting device.

In some embodiments, the controller circuit is configured to operate the external lighting device in at least two operational modes: a regular mode and a therapeutic mode. In the regular mode, the controller circuit turns on the external lighting device as general illumination. In the therapeutic mode, the controller circuit is configured to either flash the light output of the external lighting device at a frequency in a frequency range of 35˜45 Hz or generate an audible sound at a frequency in a frequency range of 35˜45 Hz, or both. The regular mode and the therapeutic mode are not necessarily mutually exclusive. For example, if a controller circuit only generates a sound at 40 Hz frequency without flashing the light output of the first light source, the controller circuit may operate the external lighting device in both the regular mode (when the first light source is on) and the therapeutic mode (when a sound at 40 Hz frequency is generated) simultaneously.

In some embodiments, the controller circuit flashes the light output of the external lighting device by turning on/off the external lighting device at a frequency in a frequency range of 35˜45 Hz.

In some embodiments, the controller circuit further comprises dimming control lines to be connected to the dimming control lines of the external lighting device such that the controller circuit flashes the light output of the external lighting device by alternating the light output level of the external lighting device between two different levels at a frequency in a frequency range of 35˜45 Hz via the dimming control lines.

In some embodiments, the controller circuit further comprises color-temperature tuning control lines to be connected to the color-temperature tuning control lines of the external lighting device such that the controller circuit flashes the light output of the external lighting device by alternating the color temperature of the external lighting device between two different color temperatures at a frequency in a frequency range of 35˜45 Hz via the color-tuning control lines.

In some embodiments, the light device further includes a sound generator. The controller circuit generates an audible sound at a frequency in a frequency range of 35˜45 Hz via the sound generator. The sound generator may be a standalone device or a component that is part of the controller circuit.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 schematically depicts images of a multi-mode antiviral air-filtering desktop lamp using white color LEDs.

FIG. 2 schematically depicts images of a multi-mode rechargeable LED flashlight.

FIG. 3 schematically depicts images of a multi-mode overbed lighting fixture.

FIG. 4 schematically depicts images of a multi-mode troffer fixture.

FIG. 5 schematically depicts images of a multi-mode lighting adaptor to a screw-in light bulb.

FIG. 6 schematically depicts an image of multi-mode lighting adaptor to an LED downlight.

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of lighting devices having different form factors.

The present disclosure discloses a multi-mode lighting device includes a visible light source configured to emit a visible light and a controlling mechanism. The controlling mechanism is configured to operate the lighting device in at least two operational modes: a regular mode and a therapeutic mode. In the regular mode, the controlling mechanism operates the visible light source for general illumination. In the therapeutic mode, the controlling mechanism is configured to either flash the visible light source at a frequency in a frequency range of 35˜45 Hz or generate an audible sound at a frequency in a frequency range of 35˜45 Hz, or both.

### Example Implementations

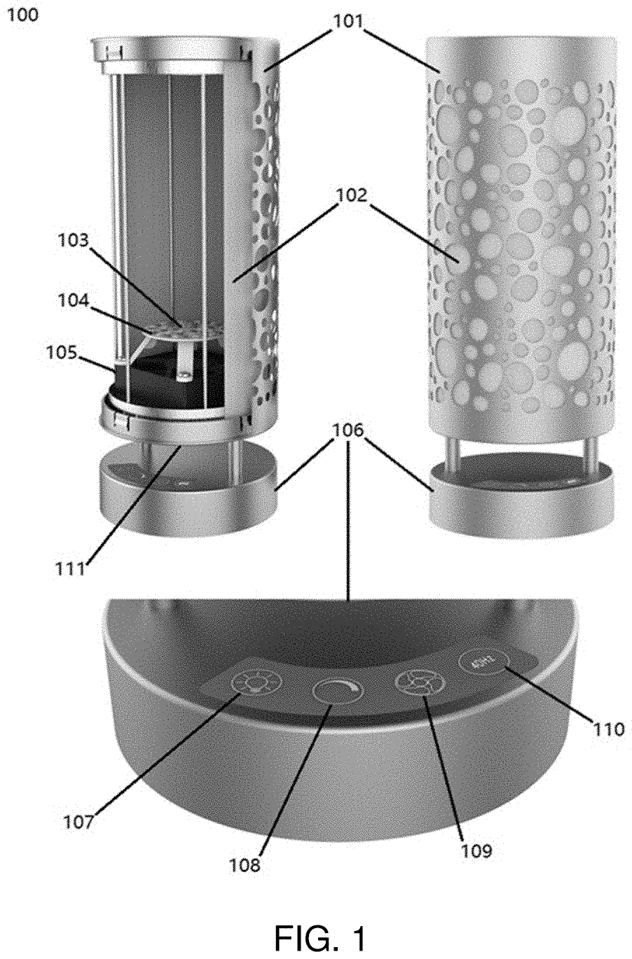

FIG. 1 is an embodiment of the lighting device of the present disclosure in a form of a desktop lamp 100. The desktop lamp 100 has an external housing 101 to house the air filter 102 and the fan 105. There are openings on the external housing 101 for the air to pass through. The fan 105 is disposed near or otherwise in a vicinity of the air inlet port 111 (e.g., disposed by or next to the air inlet port 111). The air filter 102 diffuses the light emitted by two types of visible light LEDs 103, 104. The air filter 102 is made of non-woven fabric and is coated with an antiviral photocatalytic coating (though not shown) on both interior and exterior surfaces. In some implementations, the air filter 102, as a sheet of woven fabric, may be disposed in a way that the air filter 102 curls around and shrouds (at least partially) the visible LEDs 103, 104 as well as the fan 105, without any frame or housing structure. Thus, the example shown in FIG. 1 is an optional design in which the air filter 102 is housed inside the external housing 101, although the external housing 101 may not be necessary. The antiviral photocatalyst material contains titanium dioxide (TiO₂) and nano silver. The LED light source 103 has a higher color temperature at 5000K and the LED light source 104 has a lower color temperature at 2700K. The desktop lamp 100 contains neither UV light source nor IR light source. A driver and a controller are hidden inside the base 106 of the desktop lamp 100. Through the touch button 107, the controller can switch among three color temperatures: 2700K, 3850K, and 5000K. The controller mixes 2700K LEDs and 5000K LEDs each at 50% light output to create 3850K color temperature. Through the touch button 108, the controller performs a bi-level dimming (50% and 100%) on the LED light sources 103, 104. Through the touch button 109, the controller can change the speed of the fan 105 between 50% and 100%. A light emitted by the LED light sources 103, 104 activates the antiviral photocatalytic coating on the air filter 102. The ambient air is pulled into inner chamber of the desktop lamp by the fan 105 through the air inlet port 111 and then exit out the desktop lamp through the air-permeable air filter 102 and the external housing 101. The airborne pathogens are trapped by the air filter 102 and killed by an activated photocatalyst material in the photocatalytic coating on the air filter 102.

Through the touch button 110 (with an icon “40 Hz”), the controller alternates the light output level of the LED light sources 103 and 104 between 50% and 100% at 40 Hz frequency and generate simultaneously an audible sound at 40 Hz frequency. The visual and sound stimuli induce synchronized gamma oscillations in at least one brain region of a user in the space. Since this desktop lamp 100 has 5000K and 2700K LED light sources 103, 104, another controller may be designed to alternate the color temperature of the desktop lamp between 5000K and 2700K at 40 Hz. When the touch button 110 is activated, the desktop lamp 100 operates in the therapeutic mode. When the touch button 110 is not activated, the desktop lamp 100 operates in the regular mode. The fan 105 may stay on for both modes for continuously air filtering. The photocatalytic activities remain active for both modes since the light sources 103, 104 are either fully on as in the regular mode or flashing as in the therapeutic modes, and the flashing of the light sources 103, 104 at 40 Hz can still active the antiviral photocatalyst material coated on the air filter 102.

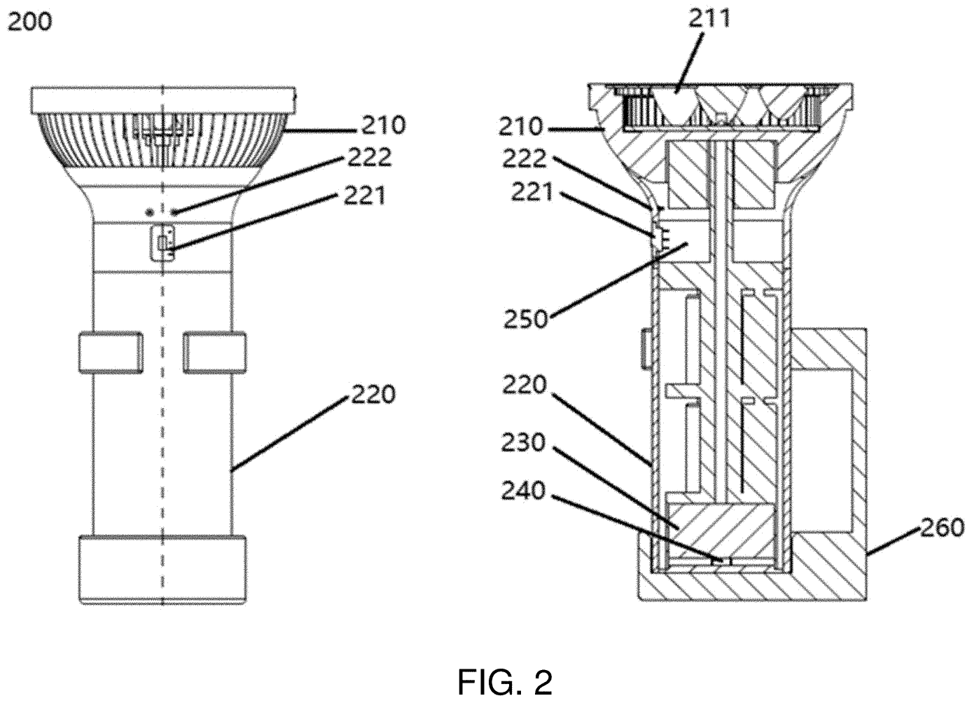

FIG. 2 is an embodiment of the lighting device of the present disclosure in a form of a multi-mode rechargeable LED flashlight 200. The flashlight 200 includes an LED lighting module 210 with multiple LED light sources 211, a housing 220, a controller 250, a rechargeable battery 230, a connector 240 for connecting the LED lighting device to an external power source, a battery charging base 260, a battery charging indicator 222, and a switch 221. When the switch 221 is switched to the top position, the controller 250 operates the flashlight 200 in the regular flashlight. When the switch 221 is switched to the bottom position, the controller 250 operates the flashlight 200 in the therapeutic mode. Under the therapeutic mode, the controller will turn on/off the LED sources 211 at 40 Hz frequency and generate simultaneously an audible sound at 40 Hz frequency.

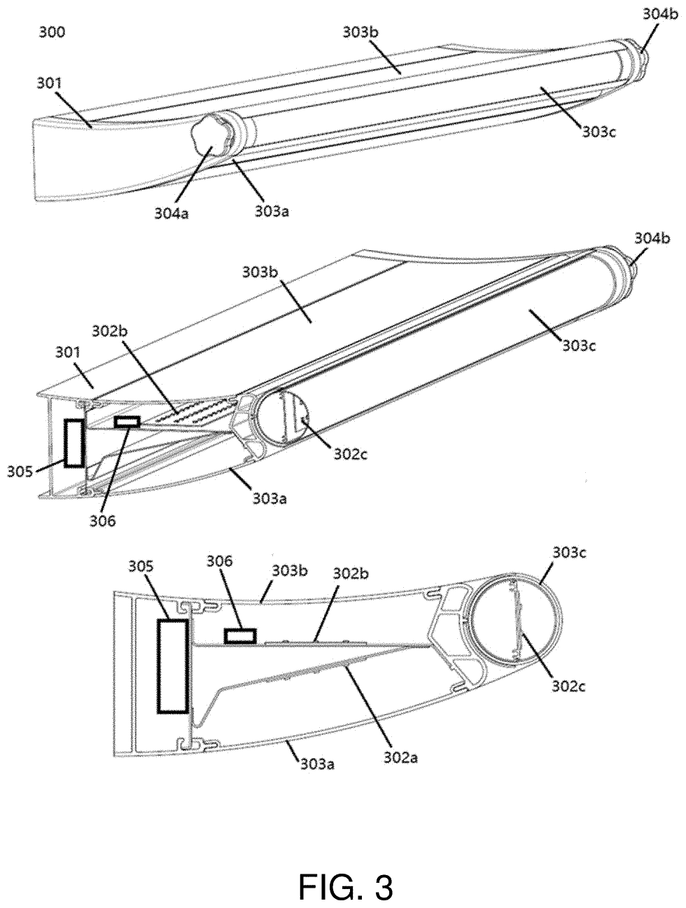

FIG. 3 is an embodiment of the lighting device of the present disclosure in a form of an overbed lighting fixture 300. The top drawing shows the exterior of the fixture, the middle drawing the interior of the fixture, and the bottom drawing the cross-section view of the fixture. The fixture 300 has a housing 301, three elongated LED lighting sources 302a, 302b, 302c, and three elongated lens 303a, 303b, 303c. The LED light sources 302a and 302b emit visible light, whereas the light source 302c is UVC LED light source emitting 280 nm wavelength. The overbed lighting fixture would be hung over the head of a patient bed. The controller 305 supports four modes: the reading mode, the ambient mode, the therapeutic mode, and the germicidal mode. In the reading mode, the controller 305 turns on the light source 302a and its light shines through the lens 303a toward the patent bed. In the ambient mode, the controller 305 turns on the light source 302b and its light shines through the lens 303b toward the ceiling. The controller 305 turns on the UVC light source 302c in the germicidal mode to provide air and surface disinfection to the patient's room. The light of the light source 302c shines through the lens 303c. The rotating knobs 304a, 304b are used to adjust the direction of the lens 303c, thus allowing a user to adjust the lighting direction of the UVC light source 302c. In the therapeutic mode, the controller 305 will turn off the UVC light source 302C and flash the light output of the light sources 302a and 302b at 40 Hz frequency (by turning on/off these light sources). Additionally, the generate controller 305 will generate simultaneously an audible sound at 40 Hz frequency via a designated sound generator 306.

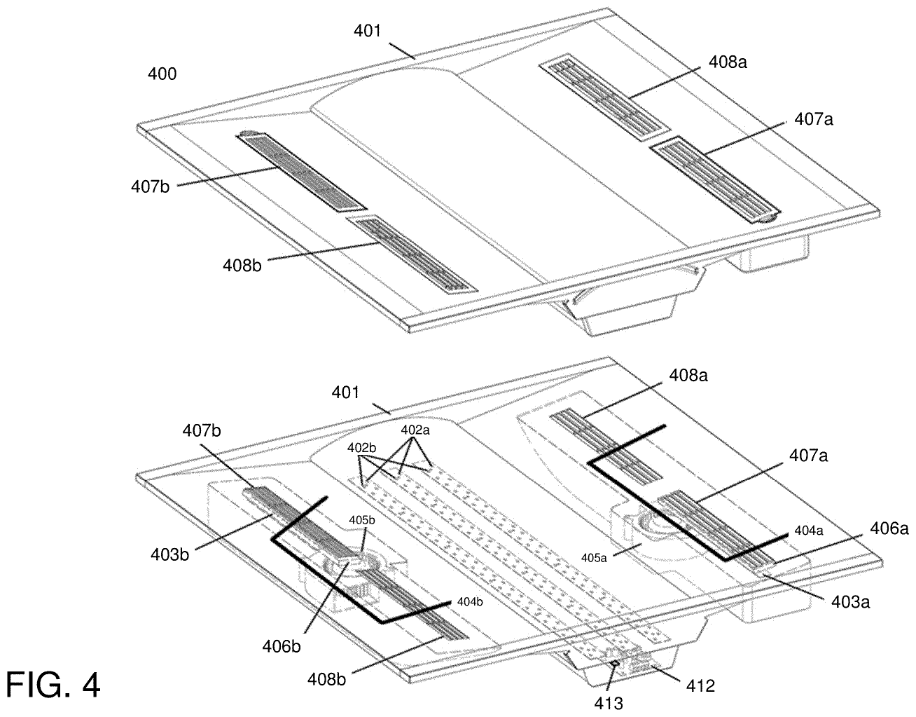

FIG. 4 shows an embodiment of the lighting device of the present disclosure in a form of an LED troffer fixture 400. This troffer has a housing 401, the first light source 402a, 402b, the second light source 403a, 403b, two airways 404a, 404b, two fans 405a, 405b and two air filters 406a, 406b. The first light source comprises six rows of LEDs on three PCBs. Out of the six rows of LEDs, three rows are 2700K LEDs 402a and the other three rows are 6500K LED 402b. The 2700K LEDs produce a lesser circadian stimulus and is more suitable for nighttime lighting, whereas the 6500K LED produce a higher circadian stimulus and is more suitable for daytime use. The combined light output of 2700K and 6500K LEDs sets the total light output of the lighting device. Since they both emit visible light, their combined light is in the visible light wavelength range. A controller 412 is used to color-tune the light output of the light device by changing the mixing ratio of the light output of the 2700K LEDs 402a and the 6500K LEDs 402b. Moreover, a memory module 413 is used to store a circadian schedule and a schedule for the therapeutic mode. The controller 412 can color-tune the light output of the first light source automatically according to the circadian schedule stored in the memory module 413. The circadian schedule will transition the color temperature of the first light source from warm white (2700K) to cold white (6500K) at dawn and revert the color temperature back to warm white at sunset, thus emulating color transition of the sunlight. The schedule may be hardcoded in the memory module 413 or be updateable from an external lighting management system.

The construction of the two airways 404a and 404b are the same, therefore the description below is on the airway 404a. The second light source 403a concealed in the housing 401 comprises multiple UV LEDs and their light outputs are confined in the lighting device. The airway 404a has an air inlet port 407a and an air outlet 408a. The fan 405a and the air filter 406a are positioned inside the airway 404a. As the fan 405a forces the air through the airway 404a, airborne microbials are trapped on the surface of the air filter 406a. The air filter 406a is coated with an antiviral photocatalyst material TiO₂, which can be adequately activated by the nearby UV LEDs 403a to kill and decompose the trapped microbials on the air filter.

As mentioned above, the memory module 413 also stores a schedule for the therapeutic mode. During the therapeutic mode, the controller 412 flashes the light output of the first light source 402a, 402b at 40 Hz by alternating the light output between 402a and 402b, and generate simultaneously an audible sound at 40 Hz frequency via one sound generating component inside the controller. The therapeutic mode operation doesn't affect the operation of the second light source 403a, 403b or the fans 405a, 405b for they are operating continuously around the clock. Therefore, the air filtering and the antiviral photocatalyst activities would continue in the therapeutic mode.

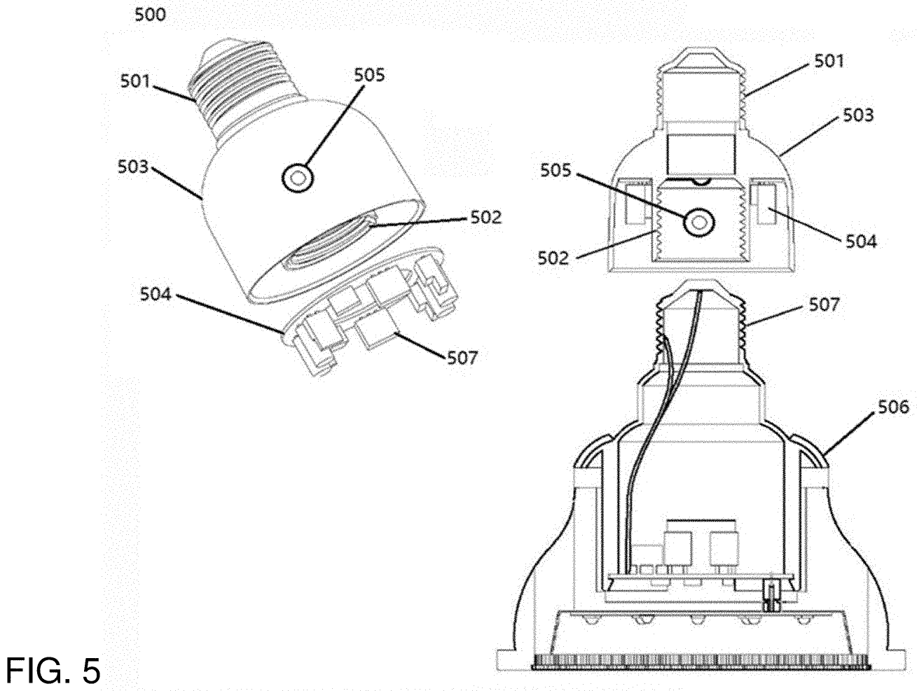

FIG. 5 shows an embodiment of the present disclosure in a form of a multi-mode lighting adaptor to a screw-in bulb. The adaptor 500 has an electrical input port 501, an electrical output port 502, a housing 503, a controller circuit 504, and a push button 505. The adaptor 500 is to be used with a screw-in bulb 506. The electrical input port 507 of the screw-in bulb 506 will be screwed into the electrical output port 502 of the adaptor 500. Before the push button 505 is pushed or activated, the controller circuit 504 allows an external AC power to be transmitted from the electrical input port 501 to the electrical output port 502 and to the electrical input port 507 of the screw-in bulb 506. When the push button 505 is activated, the controller circuit 504 flashes the light output of the light bulb 506 by turning on/off the power to the light bulb at 40 Hz. Additionally, the controller circuit 504 generates an audible sound at 40 Hz frequency via a sound generator component 507.

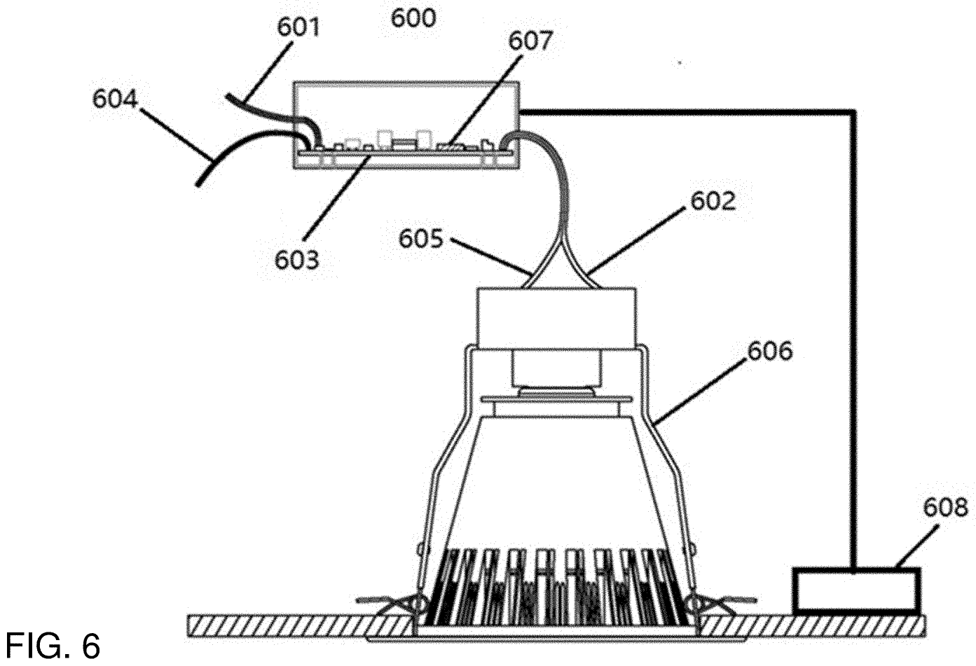

FIG. 6 shows an embodiment of the present disclosure in a form of a multi-mode lighting adaptor to an external lighting fixture. The adaptor 600 has an electrical input port 601, an electrical output port 602, a controller circuit 603, a dimming control input 604, a dimming control output 605, and a memory module 607. The electrical output port 602 and the diming control output 605 are connected to an external lighting fixture, in this case, an LED downlight fixture 606. The dimming control input 604 is connected to a dimmer on a wall to be used by a user. The memory module 607 is used to store the therapeutic schedule, where the schedule may be hardcoded in the memory or is updateable via a remote lighting management system. In the regular mode, the controller circuit 603 turns on the fixture 606 as general illumination in the space. In the therapeutic mode, the controlling circuit 603 flashes of the light output of the lighting device 606 by alternating the light output level of the external lighting device between two different levels at 40 Hz via the dimming control output 605. Additionally, the controlling circuit 603 generates an audible sound at 40 Hz frequency via a separate sound generator device 608.

### Additional and Alternative Implementation Notes

Although the techniques have been described in language specific to certain applications, it is to be understood that the appended claims are not necessarily limited to the specific features or applications described herein. Rather, the specific features and examples are disclosed as non-limiting exemplary forms of implementing such techniques.

As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A lighting device, comprising:
a first visible light source configured to emit a visible light;
an air filter coated with an antiviral photocatalyst material; and
an air circulation mechanism,
wherein, in operation:
the air circulation mechanism sucks an ambient air from outside the lighting device to force the air through the air filter;
the air filter traps airborne microbials in the air as the air passes through;
the visible light emitted by the first visible light source activates the antiviral photocatalyst material on the air filter; and
the activated antiviral photocatalyst material kills or deactivates the airborne microbials trapped on the air filter.

2. The lighting device of claim 1, further comprising:
an air inlet port,
wherein the air circulation mechanism is disposed near the air inlet port.

3. The lighting device of claim 1, wherein the first visible light source is disposed inside the air filter such that the visible light shines through the air filter to activate the antiviral photocatalyst material on the air filter, and wherein the air filter diffuses the visible light emitted from the first visible light source.

4. The lighting device of claim 1, wherein there is no ultraviolet (UV) light source or infrared (IR) light source in the lighting device.

5. The lighting device of claim 1, wherein the air filter requires no frame to house the first visible light source.

6. The lighting device of claim 1, further comprising:
a housing configured to house the air filter and the air circulation mechanism,
wherein the housing has openings thereon to allow the air to exit out of the lighting device.

7. The lighting device of claim 1, wherein the antiviral photocatalyst material coated on the air filter contains titanium dioxide (TiO₂).

8. The lighting device of claim 1, wherein the antiviral photocatalyst material coated on the air filter contains titanium dioxide (TiO₂) and at least one metal photocatalyst material such as silver, gold, copper, zinc, nickel, or a combination thereof.

9. The lighting device of claim 1, wherein the air circulation mechanism comprises a fan.

10. The lighting device of claim 1, wherein the first visible light source comprises one or more light emitting diodes (LEDs).

11. The lighting device of claim 1, wherein the first visible light source comprises a second visible light source and a third visible light source, and wherein a color temperature of the second visible light source is higher than a color temperature of the third visible light source.

12. The lighting device of claim 11, further comprising:
a color-tuning controller,
wherein the color-tuning controller is configured to tune a color temperature of the first visible light source by mixing a combination ratio of the color temperatures of the second visible light source and the third visible light source, either manually or automatically.

13. The lighting device of claim 1, further comprising:
a controlling mechanism configured to either flash a light output of the first visible light source at a frequency in a frequency range of 35˜45 Hz or generates an audible sound at a frequency in a frequency range of 35˜45 Hz, or both flash the light output and generate the audible sound.

14. The lighting device of claim 13, wherein the controlling mechanism is further configured to operate the lighting device in at least two operational modes comprising a regular mode and a therapeutic mode such that:
in the regular mode, the controlling mechanism turns on the first visible light source as general illumination, and
in the therapeutic mode, the controlling mechanism either flashes the light output of the first visible light source at the frequency in the frequency range of 35˜45 Hz or generates the audible sound at the frequency in the frequency range of 35˜45 Hz, or both flash the light output and generate the audible sound.

15. The lighting device of claim 13, wherein the controlling mechanism flashes the light output of the first visible light source by turning on and off the first visible light source at the frequency in the frequency range of 35˜45 Hz.

16. The lighting device of claim 13, wherein the controlling mechanism flashes the light output of the visible light source by alternating a light output level of the first visible light source between two different levels at the frequency in the frequency range of 35˜45 Hz.

17. The lighting device of claim 13, wherein the controlling mechanism flashes the light output of the first visible light source by alternating the color temperature of the first visible light source between two different color temperatures at the frequency in the frequency range of 35˜45 Hz.

18. The lighting device of claim 13, further comprising:
a sound generator,
wherein the controlling mechanism generates an audible sound at the frequency in the frequency range of 35˜45 Hz via the sound generator.

19. A lighting device, comprising:
a visible light source configured to emit a visible light, and
a controlling mechanism,
wherein:
the controlling mechanism is configured to either flash a light output of the visible light source at a frequency in a frequency range of 35˜45 Hz or generate an audible sound at a frequency in a frequency range of 35˜45 Hz, or both flash the light output and generate the audible sound.

20. The lighting device of claim 19, wherein the controlling mechanism is further configured to operate the lighting device in at least two operational modes comprising a regular mode and a therapeutic mode such that:
in the regular mode, the controlling mechanism turns on the first visible light source as general illumination, and
in the therapeutic mode, the controlling mechanism flashes the light output of the first visible light source at the frequency in the frequency range of 35˜45 Hz or generates the audible sound at the frequency in the frequency range of 35˜45 Hz, both flash the light output and generate the audible sound.

21. The lighting device of claim 19, wherein the controlling mechanism flashes the light output of the first visible light source by turning on and off the first visible light source at the frequency in the frequency range of 35˜45 Hz.

22. The lighting device of claim 19, wherein the controlling mechanism flashes the light output of the first visible light source by alternating the light output level of the first light source between two different levels at a frequency in a frequency range of 35˜45 Hz.

23. The lighting device of claim 19, wherein the controlling mechanism flashes the light output of the first visible light source by alternating the color temperature of the first visible light source between two different color temperatures at the frequency in the frequency range of 35˜45 Hz.

24. The lighting device of claim 19, further comprising:
a sound generator,
wherein the controlling mechanism generates the audible sound at the frequency in the frequency range of 35˜45 Hz via the sound generator.

25. The lighting device of claim 19, further comprising:
a second light source configured to emit a light in a wavelength range of 190 nm 420 nm,
wherein the controlling mechanism is further configured to operate the lighting device in at least three operational modes comprising a regular mode, a therapeutic mode, and a germicidal mode such that:
in the regular mode, the controlling mechanism turns on the first visible light source as general illumination,
in the therapeutic mode, the controlling mechanism either flashes the light output of the first visible light source at the frequency in the frequency range of 35˜45 Hz or generates the audible sound at the frequency in the frequency range of 35˜45 Hz, or both flash the light output and generate the audible sound, and
in the germicidal mode, the controlling mechanism turns on the second light source as germicidal irradiation.

26. The lighting device of claim 25, wherein a light output of the second light source is confined within the lighting device.

27. The lighting device of claim 26, wherein the controlling mechanism is further configured to always operate the lighting device in the germicidal mode.

28. A lighting device adaptor, comprising:
an electrical input port;
an electrical output port; and
a controller circuit,
wherein:
the electrical input port is configured to connect to an external power source,
the electrical output port is configured to connect to an electrical input port of a lighting device to provide power to the external lighting device, and
the controller circuit is configured to either flash a light output of the lighting device at a frequency in a frequency range of 35˜45 Hz or generate an audible sound at a frequency in a frequency range of 35˜45 Hz, or both flash the light output and generate the audible sound.

29. The lighting device adaptor of claim 28, wherein the controller circuit is further configured to operate the lighting device in at least two operational modes comprising a regular mode and a therapeutic mode such that:
in the regular mode, the controller circuit turns on the lighting device as general illumination, and
in the therapeutic mode, the controller mechanism either flashes the light output of the lighting device at the frequency in the frequency range of 35˜45 Hz or generates the audible sound at the frequency in the frequency range of 35˜45 Hz, or both flashes the light output and generates the audible sound.

30. The lighting device adaptor of claim 28, wherein the controller circuit flashes the light output of the lighting device by turning on and off the lighting device at the frequency in the frequency range of 35˜45 Hz.

31. The lighting device adaptor of claim 28, wherein the controller circuit comprises dimming control lines connected to dimming control lines of the lighting device, and wherein the controller circuit flashes the light output of the lighting device by alternating a light output level of the lighting device between two different levels at the frequency in the frequency range of 35˜45 Hz via the dimming control lines of the controller circuit.

32. The lighting device adaptor of claim 28, wherein the controller circuit comprises color-temperature tuning control lines connected to color-temperature tuning control lines of the lighting device, and wherein the controller circuit flashes the light output of the lighting device by alternating a color temperature of the lighting device between two different color temperatures at the frequency in the frequency range of 35˜45 Hz via the color-tuning control lines.

33. The lighting device adaptor of claim 28, further comprising:
a sound generator,
wherein the controller circuit generates the audible sound at the frequency in the frequency range of 35˜45 Hz via the sound generator.
